(19) United States
(12) Patent Application Publication
(10) Pub. No.: US 2012/0271202 A1
(43) Pub. Date: Oct. 25, 2012

(54) ULTRASONIC THERAPY DEVICE WITH DIFFRACTIVE FOCUSING

(75) Inventor: Jeffrey Alan WISDOM, Brisbane, CA (US)

(73) Assignee: Cutera, Inc., Brisbane, CA (US)

(21) Appl. No.: 13/426,566

(22) Filed: Mar. 21, 2012

Related U.S. Application Data
(60) Provisional application No. 61/466,847, filed on Mar. 23, 2011.

(51) Int. Cl. A61N 7/00

(52) U.S. Cl. 601/2

(57) ABSTRACT

A device for selectively treating multiple regions of tissue simultaneously, the device comprising an ultrasound transducer and a plurality of layers. The ultrasound transducer is configured to produce an acoustic wave. The plurality of layers comprises a plurality of cavities, where the plurality of cavities are configured to scatter the acoustic wave and simultaneously produce a plurality of treatment zones at a predetermined distance from the ultrasound transducer.

### CROSS-REFERENCE TO RELATED APPLICATIONS

This application claims priority to U.S. Provisional Application No. 61/466,847 filed Mar. 23, 2011, which is incorporated herein in its entirety for all purposes.

### BACKGROUND

1. Field

The present application relates generally to ultrasound transducers, and more particularly to a method and apparatus for selectively treating multiple regions of tissue simultaneously.

2. Related Art

There has been considerable interest in using ultrasonic energy to treat various medical or dermatological conditions. For example, ultrasound energy has been used for hair removal. In addition, ultrasound energy has been used to improve skin conditions and reduce fat or cellulite.

Ultrasound energy is often delivered via a handpiece carrying one or more ultrasound transducers. In some cases, the ultrasound energy is unfocused, while in others, the energy is focused. Various approaches have been used for focusing the ultrasound energy.

In general, an ultrasound device may be characterized as a device capable of producing displacements at a frequency higher than the audible range of a human ear (frequencies>20,000). Ultrasound devices typically include a transducer that converts electrical energy into acoustical energy via vibrational motion at ultrasonic frequencies. The ultrasound vibration is induced by exciting one or more piezoelectric elements of the transducer using an electrical signal

The following publications relate to ultrasound treatment devices and are commonly owned by the assignee of the present application: U.S. Pat. App. Pub. 2008/0195000, U.S. Pat. App. Pub. 2009/0171253, U.S. Pat. App. Pub. 2008/0183110, U.S. Pat. App. Pub. 2010/0126275, U.S. Pat. App. Pub. 2010/0211060, and U.S. Pat. App. Pub. 2010/0249670. Each of the foregoing are incorporated herein by reference in their entireties.

The benefits of providing multiple treatments zones are well-known. Known devices for producing multiple treatment zones are disclosed in the following publications, each of which is incorporated herein by reference. U.S. Pat. No. 6,997,923, U.S. Pat. No. 7,331,953, U.S. Pat. App. Pub., 2003/0216719, U.S. Pat. App. Pub. 2007/0239079, and U.S. Pat. App. Pub. 2006/0155266.

Traditional efforts to achieve multiple treatment zones use a single focused ultrasound source that was translated around the patient to introduce thermal damage. Such methods and systems are complicated and expensive to manufacture, while being difficult and time consuming to operate.

Other traditional methods and systems of providing multiple treatment zones have used multiple lens and/or transducers, each providing a single treatment zone. Such methods and systems are also complicated and expensive to manufacture.

In the present application, improved ultrasound transducers are disclosed. The ultrasound transducers disclosed herein produce multiple simultaneous treatment zones without complicated and expensive equipment.

### SUMMARY

In one embodiment, a device for selectively treating multiple regions of tissue simultaneously, the device comprising an ultrasound transducer and a plurality of layers. The ultrasound transducer is configured to produce an acoustic wave. The plurality of layers comprises a plurality of cavities, where the plurality of cavities are configured to scatter the acoustic wave and simultaneously produce a plurality of treatment zones at a predetermined distance from the ultrasound transducer. In a further embodiment, the plurality of cavities are configured to produce at least one of the plurality of treatment zones by constructive interference of at least two portions of the acoustic wave that are scattered by a respective two of the plurality of cavities.

In another embodiment, the plurality of layers comprises one selected from the group consisting of: a piezoelectric source, a wearplate, and a transmissive layer. In a further embodiment, the plurality of cavities comprises a plurality of etchings within the layer. In yet another embodiment, the one of the plurality of layers comprises the transmissive layer and the plurality of cavities comprise a material different from a material of the transmissive layer.

In another embodiment, the cavities are arranged to create a pattern. In a further embodiment, the pattern is one selected from a hexagonal symmetry and a square symmetry.

In another embodiment, at least one of the plurality of cavities comprises a circular shape. In yet another, at least one of the cavities comprises a polygonal shape. In a further embodiment, the polygonal shape is one selected from the group consisting of a square and a hexagon. In yet a further embodiment, the cavities are arranged to create a pattern, wherein the pattern is one selected from a hexagonal symmetry and a square symmetry.

In another embodiment, at least one of a size and a position of each of the cavities is optimized for a predetermined application. In a further embodiment, the predetermined application comprises at least one of a predetermined skin type and a predetermined treatment. In yet a further embodiment, the predetermined treatment comprises a calculation of the predetermined distance. In another embodiment, the at least one of the size and the position comprises selecting a distance between cavities.

In another embodiment, an ultrasound device for selectively treating multiple regions of tissue simultaneously comprises an ultrasound transducer assembly. The assembly comprises a planar delivery surface through which ultrasound energy is transmitted and a planar region located between the delivery surface and the tissue. The planar region comprises a periodic pattern of alternating higher and lower transmission regions, which function to cause interference in ultrasonic waves and cause the ultrasound energy to be focused at multiple regions in a plane spaced from the transducer.

In a further embodiment, the planar region is formed integrally with the transducer. In another embodiment, the planar region is formed in a separate layer bonded to the transducer. In yet another embodiment, the periodic pattern is formed by openings in the layer.

### BRIEF DESCRIPTION OF THE FIGURES

The present application contains at least one drawing in color format. Copies of this patent or patent application publication with color drawing(s) may be provided by the Office upon request and payment of the necessary fee.

The present application can be best understood by reference to the following description taken in conjunction with the accompanying figures, in which like parts may be referred to by like numerals.

FIG. 1 is a schematic illustration of an ultrasound transducer in accordance with an exemplary embodiment of the invention.

FIG. 2a illustrates a top view of an incident pressure field in accordance with an exemplary embodiment of the invention.

FIG. 2b illustrates a top view of a treatment pressure field in accordance with an exemplary embodiment of the invention.

FIG. 3a illustrates a cross-sectional view of an ultrasound transducer comprising a PZT layer and an aluminum interface in accordance with an exemplary embodiment of the invention.

FIG. 3b illustrates a cross-sectional view of an ultrasound transducer comprising a PZT layer and an aluminum interface in accordance with an exemplary embodiment of the invention.

FIG. 3c illustrates a cross-sectional view of an ultrasound transducer comprising a PZT layer and an aluminum interface in accordance with an exemplary embodiment of the invention.

FIG. 3d illustrates a cross-sectional view of an ultrasound transducer comprising a PZT layer, an aluminum interface, and superficial absorbers in accordance with an exemplary embodiment of the invention.

FIG. 3e illustrates a cross-sectional view of an ultrasound transducer comprising a PZT layer, an aluminum interface, and a transmissive layer in accordance with an exemplary embodiment of the invention.

FIG. 3f illustrates a cross-sectional view of an ultrasound transducer comprising a PZT layer, an aluminum interface, a first transmissive layer, and a second transmissive layer in accordance with an exemplary embodiment of the invention.

FIG. 3g illustrates a cross-sectional view of an ultrasound transducer comprising a PZT layer, an aluminum interface, a first transmissive layer, and a second transmissive layer in accordance with an exemplary embodiment of the invention.

FIG. 4 illustrates a top view of a cavity pattern on an ultrasound transducer using any of the positions mentioned with respect to FIGS. 3a-3g above.

FIG. 5a illustrates a side view of a pressure distribution resulting from an ultrasound transducer with a patterned layer for scattering an acoustic wave, in accordance with an exemplary embodiment of the invention.

FIG. 5b illustrates a side view of a pressure distribution resulting from an ultrasound transducer with a patterned layer for scattering an acoustic wave, in accordance with an exemplary embodiment of the invention.

FIG. 6a illustrates a top view of an incident pressure field in accordance with an exemplary embodiment of the invention.

FIG. 6b illustrates a top view of the acoustic field of FIG. 6a after the field has propagated 1.5 mm.

FIG. 7 illustrates exemplary shapes and patterns for cavities in a patterned layer in accordance with an exemplary embodiment of the invention.

FIG. 8 illustrates an ultrasound transducer with a “diced” PZT layer.

FIG. 9 illustrates a process of manufacturing an ultrasound transducer in accordance with an exemplary embodiment of the invention.

FIG. 10a illustrates a top view of the pressure field of the ultrasound transducer of FIG. 9 at a distance of 9 mm from the front surface of the transducer.

FIG. 10b illustrates a top view of the pressure field of the ultrasound transducer of FIG. 9 at a distance of 12 mm from the front surface of the transducer.

FIG. 11 illustrates another process of manufacturing an ultrasound transducer in accordance with an exemplary embodiment of the invention.

FIG. 12 illustrates an ultrasound transducer system in accordance with an exemplary embodiment of the invention.

### DETAILED DESCRIPTION

The following description sets forth numerous specific configurations, parameters, and the like. It should be recognized, however, that such description is not intended as a limitation on the scope of the present application, but is instead provided as a description of exemplary embodiments.

Broadly, this disclosure describes systems and methods for diffracting or scattering an acoustic wave to produce constructive interference at multiple treatment zones. The constructive interference of the scattered wave results in high-intensity, localized treatment.

In some embodiments, an ultrasound transducer includes a piezo-electric source (PZT), driven by an electric voltage, with at least one layer of patterned and heterogeneous materials that scatters the ultrasound beam to create multiple focal zones at a target treatment depth.

In some embodiments, the cavities are included in the PZT. In other embodiments, the cavities are included in a wearplate which is attached to the PZT. In yet other embodiments, the cavities are included in one or more transmissive layers which may be attached to the PZT or to the wearplate. In some embodiments, the cavities in the layer form an apodized layer.

In some further embodiments, the cavities have a predetermined shape and are arranged in predetermined patterns to produce a desired depth and intensity of the treatment zones.

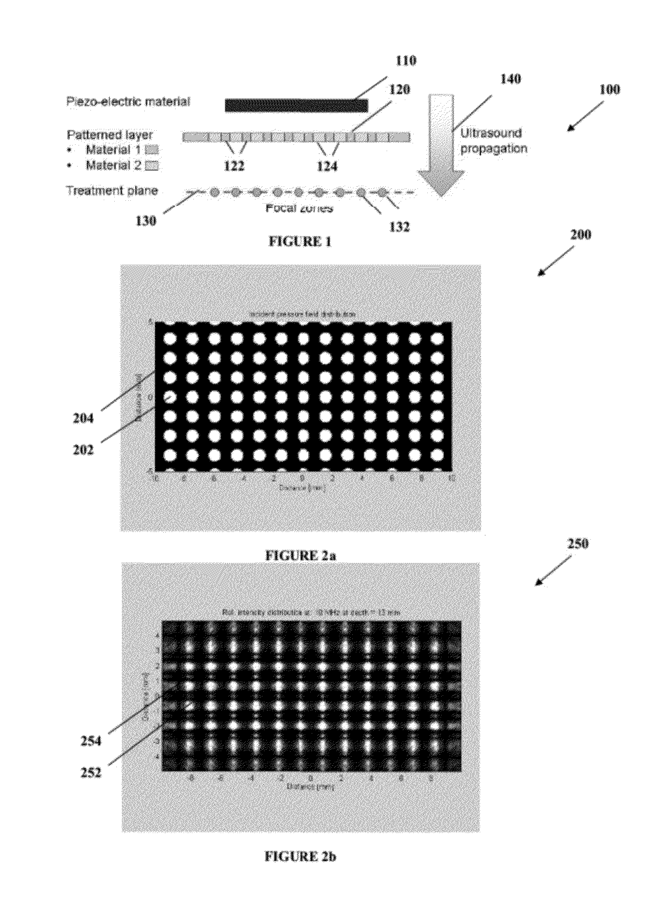

FIG. 1 is a schematic illustration of an ultrasound transducer 100 in accordance with an exemplary embodiment of the invention. Ultrasound transducer 100 includes a layer of piezoelectric material 110 and a patterned layer 120. Piezoelectric material 110 generates an acoustic field (not shown) which propagates in the direction of arrow 140.

The acoustic field generated by piezoelectric material 110 is scattered by patterned layer 120. Patterned layer 120 comprises first and second materials arranged linearly so to create a spatially varying transmission coefficient. The spatially varying transmission coefficient produces a sharp change in intensity in a transverse direction of the layer. The sharp change in intensity results in diffraction of the acoustic field, causing scattering (not shown) of the ultrasound wave.

The scattered ultrasound wave converges into a plurality of focal zones 132 which arrange linearly to form treatment plane 130. In other words, the patterned layer 120 diffracts the ultrasound wave, which then constructively interferes at the focal zones 132 to form treatment plane 130. This constructive interference generates higher intensities in the focal zones then in the surrounding tissue.

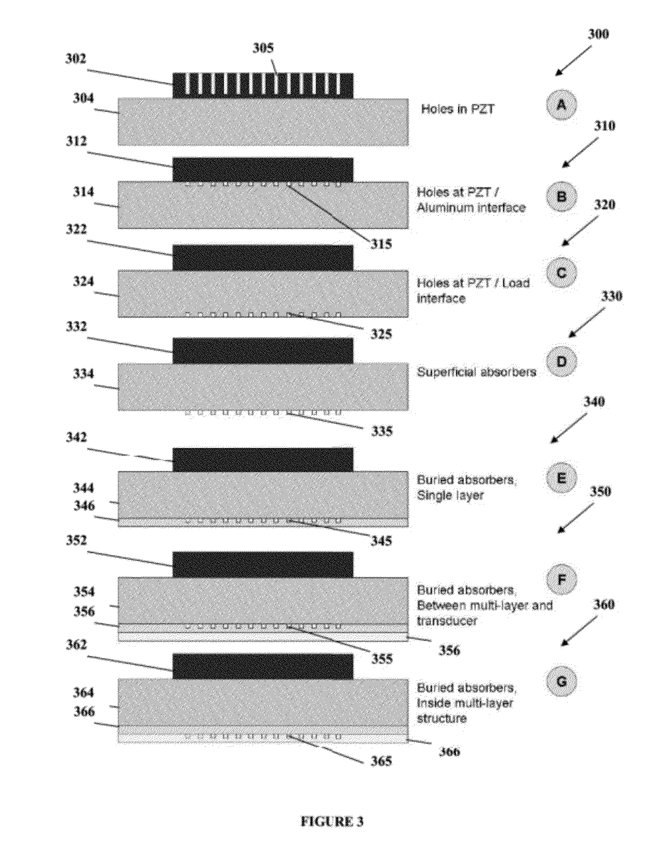

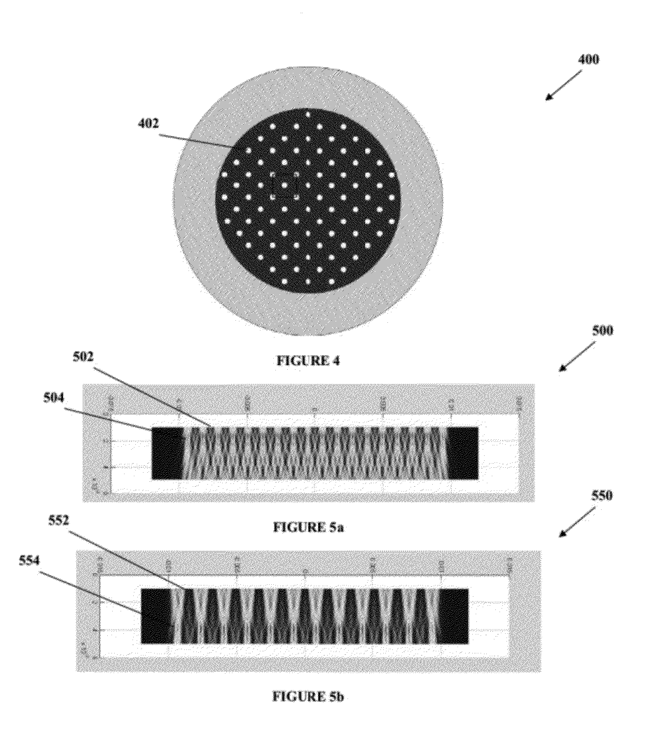

In the embodiment of FIG. 1, piezoelectric material 110 and patterned layer 120 are illustrated as detached. In some embodiments, additional layers may be positioned between piezoelectric material 110 and patterned layer 120. In other embodiments, piezoelectric material 110 and patterned layer 120 may be attached or bonded together. In still further embodiments, piezoelectric material 110 may comprise the patterned layer, as described more fully below with respect to the embodiment of FIG. 3a.

The embodiment of FIG. 1 depicts first and second materials in the patterned layer. In some embodiments, the patterned layer comprises a first material and air. In such embodiments, the patterned layer 120 comprises a series of holes or etchings within the layer. Other embodiments may comprise a third material configured to provide an additional spatial variance in the transmission coefficient, which may be positioned within the patterned layer to create a varying intensity of focal zones across a treatment plane.

FIG. 2a illustrates a top view of an incident pressure field 200 in accordance with an exemplary embodiment of the invention. Incident pressure field 200 may represent the intensity of an acoustic field as it exits a patterned layer, such as patterned layer 120 discussed above with reference to FIG. 1. Increasing intensity of the acoustic field is depicted by an increasing shade of white. That is, the white circles 202 in FIG. 2a represent regions of relatively high intensity and the dark areas 204 represent regions of relatively low intensity. As can be seen in FIG. 2a, the intensity distribution extends in a two-dimensional array. The intensity of each circular area 202 is the same, which indicates a uniform pattern in the patterned layer.

FIG. 2b illustrates a top view of a treatment pressure field 250 in accordance with an exemplary embodiment of the invention. In the embodiment of FIG. 2b, the treatment pressure field 250 is located at a distance of 13 mm from the incident pressure field shown in FIG. 2a. As with FIG. 2a above, increasing intensity is represented by an increasing shade of white. Treatment pressure field 250 may represent the intensity of an acoustic field in a treatment plane, such as treatment plane 130 described above with reference to FIG. 1. Treatment pressure field 250 may be produced when an acoustic field scatters from a patterned layer, such as patterned layer 120 discussed above with reference to FIG. 1. As can be seen in FIG. 2b, the intensity is relatively high at a plurality of focal zones 252 within the treatment plane. The array of focal zones may be used to selectively heat regions of tissue. Additional zones 254 are also present and may or may not provide a therapeutic benefit to the tissue. Zones 254 are also produced by the scattering of the acoustic wave, but result in a focal zones of lower intensity. Although the treatment pressure field 250 is located at a distance of 13 mm from the pressure field, in other embodiments, an ultrasound transducer is configured to form a treatment pressure field at another distance. Such configurations may include variations of size and spacing of the elements of the patterned layer or may include variations of the position of the patterned layer, as described in more detail below.

FIGS. 3a-3g illustrate various arrangements of ultrasound transducers 300, 310, 320, 330, 340, 350, and 360 in accordance with exemplary embodiments of the invention. FIGS. 3a-3g illustrate various cavity positions in various combinations of PZT layers, wearplates, and transmissive layers.

Each of transducers 300, 310, 320, 330, 340, 350, and 360 is assumed to operate on a mechanical resonance in the vertical direction by a sinusoidal voltage (or sum of different sinusoidal voltages) applied to the PZT. The embodiments of FIGS. 3a-3g assume a PZT layer with an aluminum interface. There are a number of different ultrasonic transducers which would have different material implementations, but could utilize the structure described herein. In some embodiments, the aluminum thickness is chosen to be a (n/2+¼)*λ thickness for one of the resonant frequencies of the aluminum/pzt combination (n is an integer). However, other thicknesses can be chosen to lower resonating fields or higher resonating fields inside the transducer.

Although aluminum is depicted as the wearplate in each of FIGS. 3a-3g, it will readily be understood by a person of ordinary skill in the art that any suitable metal can be used as the wearplate without deviating from the scope of the invention. Such suitable materials include, but are not limited to, biocompatible materials, such as titanium. The wearplate may act as an acoustic matching layer, allowing more efficient transmission of ultrasound energy. The wearplate may also provide an electrical connection to one side of the PZT. The wearplate may also provide mechanical stability to the PZT layer.

FIG. 3a illustrates a cross-sectional view of an ultrasound transducer 300 comprising a PZT layer 302 and an aluminum interface 304 in accordance with an exemplary embodiment of the invention. PZT layer 302 comprises cavities 305 for scattering an ultrasound wave produced by ultrasound transducer 300.

As used herein, a cavity of a layer can be understood to describe an empty volume, a hole, or a region in the layer that includes a material different than the material in the layer, for example. In each case, the cavity creates a linear variation in spatial transmission coefficient, which scatters the acoustic wave, as explained in more detail below. Such a cavity could be termed a “defect” of the layer. When the cavity is filled with another material different from the material of the layer, the other material may be termed an “absorber.”

FIG. 3b illustrates a cross-sectional view of an ultrasound transducer 310 comprising a PZT layer 312 and an aluminum interface 314 in accordance with an exemplary embodiment of the invention. Aluminum interface 314 comprises cavities 315 at the PZT interface for scattering an ultrasound wave produced by ultrasound transducer 310.

FIG. 3c illustrates a cross-sectional view of an ultrasound transducer 320 comprising a PZT layer 322 and an aluminum interface 324 in accordance with an exemplary embodiment of the invention. Aluminum interface 324 comprises cavities 325 at the load interface (skin interface) for scattering an ultrasound wave produced by ultrasound transducer 320.

FIG. 3d illustrates a cross-sectional view of an ultrasound transducer 330 comprising a PZT layer 332, an aluminum interface 334, and superficial absorbers 335 in accordance with an exemplary embodiment of the invention. Superficial absorbers are configured to scatter an ultrasound wave produced by ultrasound transducer 330.

FIG. 3e illustrates a cross-sectional view of an ultrasound transducer 340 comprising a PZT layer 342, an aluminum interface 344, and a transmissive layer 346 in accordance with an exemplary embodiment of the invention. Transmissive layer 346 comprises absorbers 345 for scattering an ultrasound wave produced by ultrasound transducer 340.

Applying a coating that has a spatially varying transmission coefficient, such as in ultrasound transducer 340, may be the most direct method to generate a beam that will naturally generate foci when it diffracts. The design of this transducer is straightforward since most large-area transducers produce a flat phase profile.

Further, although FIGS. 3a-3g illustrate the PZT layers, the wearplates, and the transmissive layers as planar, persons of skill in the art will recognize that the invention is not so limited. A planar PZT layer, however, may allow for a reduced cost of manufacture. In one embodiment with a planar delivery surface, an ultrasound device for selectively treating multiple regions of tissue simultaneously comprises an ultrasound transducer assembly. The assembly comprises the planar delivery surface through which ultrasound energy is transmitted and a planar region located between the delivery surface and the tissue. The planar region comprises a periodic pattern of alternating higher and lower transmission regions, which function to cause interference in ultrasonic waves and cause the ultrasound energy to be focused at multiple regions in a plane spaced from the transducer. In some embodiments, the planar region is formed integrally with the transducer. In other embodiments, the planar region is formed in a separate layer bonded to the transducer. In some more embodiments, the periodic pattern is formed by openings in the layer.

The transmissive layer 346 may be added to adjust the focal depth of a given transducer. For example, the prototypical transducer is made from a piezoelectric material with a series of plates to engineer the output power and the operating frequency of the stack. Consider two 10-MHz transducers, one with a focal depth of 1.3 mm and another with a focal depth of 2.5 mm. In the embodiments of FIGS. 3e-3g, the external series of layers may be added to the 10-MHz transducer to allow it to focus at either 1.3 or 2.5 mm. In this way, the production of the transducers is decoupled from the focal depth required, rather than holding two separate inventories of PZTs and plates with different cavity pitches for the desired foci and operating frequency. This may provide a low-cost option for patient-specific applications of the ultrasound transducers described herein.

FIG. 3f illustrates a cross-sectional view of an ultrasound transducer 350 comprising a PZT layer 352, an aluminum interface 354, a first transmissive layer 356, and a second transmissive layer 358 in accordance with an exemplary embodiment of the invention. Transmissive layer 356 comprises absorbers 355 for scattering an ultrasound wave produced by ultrasound transducer 350.

FIG. 3g illustrates a cross-sectional view of an ultrasound transducer 360 comprising a PZT layer 362, an aluminum interface 364, a first transmissive layer 366, and a second transmissive layer 368 in accordance with an exemplary embodiment of the invention. Transmissive layer 368 comprises absorbers 365 for scattering an ultrasound wave produced by ultrasound transducer 360.

FIG. 4 illustrates a top view of a cavity pattern on an ultrasound transducer 400 using any of the designs mentioned with respect to FIGS. 3a-3g above. The cavities are positioned such that the scattered waves from their edges constructively interfere at a predetermined position. For example, consider holes arranged in a periodic, 2D array in the transducers described above in FIGS. 3a-3g. That is, holes 402 may correspond to cavities 305, 315, 325, 335, 345, 355, and 365 discussed above.

The hole array pattern will influence the field distribution in the plane of interest. Also, the separation distance from hole to hole will influence the depth plane for the focal spots. A smaller separation distance will result in focal spots closer to the transducer, while separating them pushes back the focal plane. The separation scale is indicated with a black square.

FIGS. 5a and 5b illustrate a side view of a pressure distribution resulting from an ultrasound transducer with a patterned layer for scattering an acoustic wave, in accordance with an exemplary embodiment. In both images, the plane of the occlusions is at the “zero” point at the top of the image. As can be seen in FIGS. 5a and 5b, the acoustic wave is scattered at the edges 502 and 552 of the cavities, which causes focal zones 504 and 554 downstream.

The center-to-center separations of the cavities are varied between FIGS. 5a and 5b. The x and y dimensions are in meters. In FIG. 5a, the pitch is 1.1 mm, and the foci are located at around 2 mm down from the location of the plane of the occlusions. In the FIG. 5b, the pitch is 1.7 mm and the foci are located 4 mm down from the plane of the occlusions.

When used in the body, the diffracted field will be influenced by absorption in tissue. Attenuation reduces the peak field experienced at the targeted depth. For example, using a 2D array of cavities with hexagonal symmetry there may be no peak intensity higher than 1.5 times the input intensity at a depth between 1 to 3 mm. Having only a few cavities that provide scattered edge waves limits the effective focal gain of the device (the amount of power at the focus relative to the input power, which may be particularly important in the presence of attenuation and the desire to deposit heat at a particular depth).

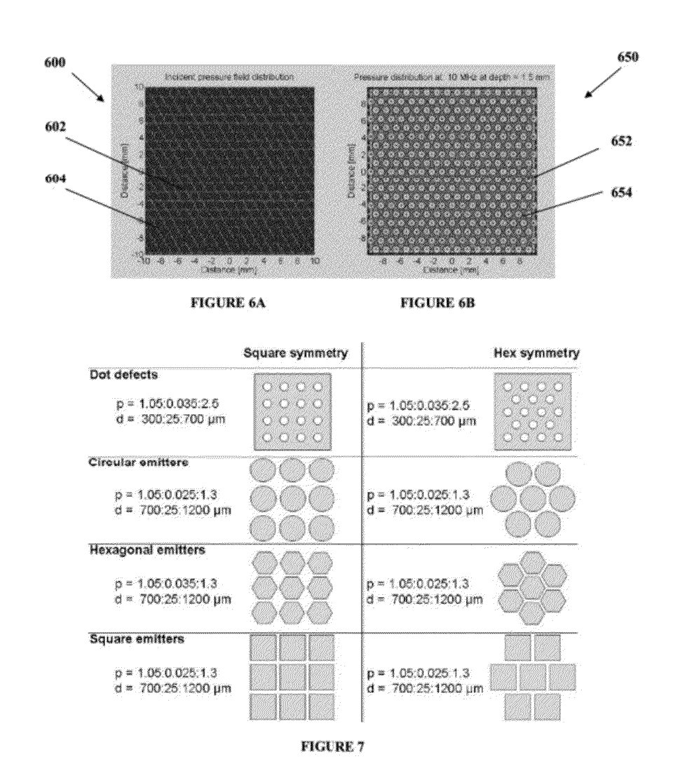

FIG. 6a illustrates a top view of an incident pressure field 600 in accordance with an exemplary embodiment of the invention. In FIG. 6a, blue represents no-field and red (circles) indicates the nominal incident pressure. The cavity pattern is such that the source regions look like circles in a hexagonal, close-packed orientation. FIG. 6b is a top view of the acoustic intensity versus position after the field of FIG. 6a has propagated 1.5 mm. The frequency is 10 MHz, and an attenuation of 28 dB/cm/MHz is assumed for the attenuation. The peak acoustic intensity is twice the incident at this location. This embodiment may provide an improvement in the focal gain over an array of cavities—such as those described above with respect to FIG. 5—from the larger perimeter of the cavity area, contributing more edge-diffraction effects.

In some embodiments, the cavities are sized sufficiently relative to the wavelength of the ultrasonic radiation to introduce a dark region in the field that will propagate to the desired depth. For example, in aluminum, a cavity that is 50 microns in diameter is less than 1/10th of the wavelength of an ultrasonic field resonating at 10 MHz. This is relatively small. An improved size range may be on the order from ⅕th of a wavelength to 1.5 wavelengths. As the size of the cavity increases, it will still produce a focal zone, but may reduce the average power delivered by the device to the patient.

FIG. 7 illustrates exemplary shapes and patterns for cavities in a patterned layer in accordance with an exemplary embodiment of the invention. The white areas in each figure represent an occlusion or etching in a layer and the blue areas represent the unmodified portion of the layer. The first column depicts various cavities in a square symmetry and the second column depicts various cavities in a hexagonal symmetry. The first row depicts circular cavities, the second row depicts circular emitters (islands), the third row depicts hexagonal emitters, and the fourth row depicts square emitters.

FIG. 8 illustrates an ultrasound transducer with a “diced” PZT layer 810. The diced pattern is created by forming linear etchings 812 and 814. The etchings are perpendicular, resulting in square emitters 816. Similar arrangements may be used in the wearplate. In one embodiment, aluminum may provide a low-cost fabrication alternative. Using rolled aluminum sheet as the wearplate, the pattern can be created using a volume-scalable photo-chemical etching process. The reduced manufacturing cost may enable a disposable layer for patient-specific skin treatments.

As described above, there are a variety of different cavity patterns and shapes which could be used to create an ultrasound transducer within the scope of the invention. Further, in accordance with some embodiments of the invention, the patterns and shapes are optimized for particular applications, such as for a particular patient or treatment type. For example, an ultrasound transducer in accordance with the present invention may be engineered to produce foci at different depths by adjusting the pitch of the occluding features (i.e., cavity-to-cavity spacing). With a field-replaceable transducer it would be possible for the practitioner to treat at different depths using the same power supply unit.

In addition, a patient's skin type may factor into the optimization of an ultrasound transducer in accordance with the invention. For example, the authors of “In Vivo High-frequency Ultrasonic Characterization of Human Dermis,” (Guittet, et al., Biomedical Engineering, June 1999), the entirety of which is incorporated by reference herein for all purposes, found that there is a large variation in the skin attenuation coefficient with age. Indeed, even with in an age group, there is significant variation in attenuation in the skin. This variation in attenuation may be used to provide an optimal treatment parameter for a particular patient. For example, the skin attenuation for a particular patient may first be measured and then correlated within a skin type range. Each skin type range may correspond to a particular cavity pattern or shape, or may correspond to a particular frequency. Further, the desired treatment, including depth of treatment, may also affect the determination of the optimal pattern, shape, or frequency.

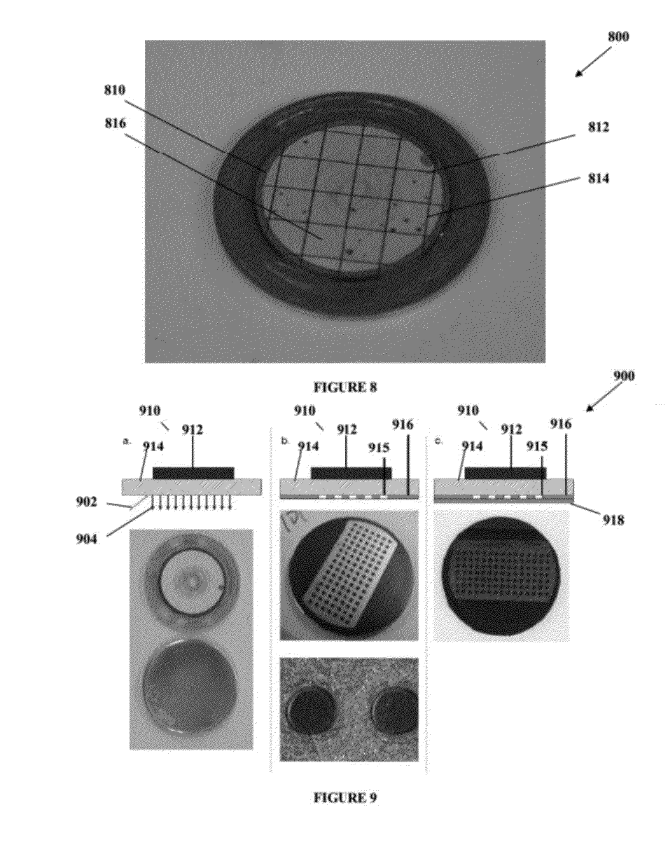

FIG. 9 illustrates a process 900 of manufacturing an ultrasound transducer in accordance with an exemplary embodiment of the invention. It should be appreciated that process 900 may include any number of additional or alternative tasks. The tasks shown in FIG. 9 need not be performed in the illustrated order and process 900 may be incorporated into a more comprehensive procedure or process having additional functionality not described in detail herein.

FIG. 9 includes a schematic (top) and photos (bottom) of an exemplary ultrasound transducer 910 at three steps in the manufacturing process. Process 900 produces an ultrasound transducer 910 with a PZT layer 912, an aluminum layer 914, a first transmissive layer 916, and a second transmissive layer 918. First transmissive layer 916 includes a series of etchings 915 configured to induce scattering of an ultrasound wave (not shown) created by the PZT. In other words, the approach here for producing an apodized beam is a coating that has a spatially varying transmission coefficient.

Although aluminum is depicted as the wearplate in FIG. 9, it will readily be understood by a person of ordinary skill in the art that any suitable metal can be used as the wearplate without deviating from the scope of the invention. Such suitable methods include, but are not limited to, biocompatible materials, such as titanium.

FIG. 9a depicts a cross sectional view of the assembled transducer prior to adding the transmissive layer. The acoustic field is released from the side indicated by green arrow 902 (referred to as the “output side”) and in the direction of the blue arrows 904.

After the PZT 912 and aluminum 914 are firmly attached, a first transmissive layer 916 is placed on the output side. The first transmissive layer 916 may include Kapton, parlyene, or any material that can (1) be easily layered and (2) withstand high average output powers. After the first transmissive layer 916 is attached, the layer is laser machined, or otherwise etched, to generate a pattern. This is shown in FIG. 9b. The non-etched portions of the first transmissive layer 916 represent the transmission pattern for the ultrasonic field. The bottom photo of FIG. 9b offers a close-up of two circular emitters in the first transmissive layer shown in the top photo of FIG. 9b.

After the pattern is successfully laser machined, a second transmissive layer 918 is placed over the first layer, as is shown in FIG. 9c. The air gaps 915 between the aluminum layer 914 and the second transmissive layer 918 act as ultrasound reflectors. In one embodiment, the pattern consists of a series of 820-micron circles with a center-to-center spacing of 1.5 mm.

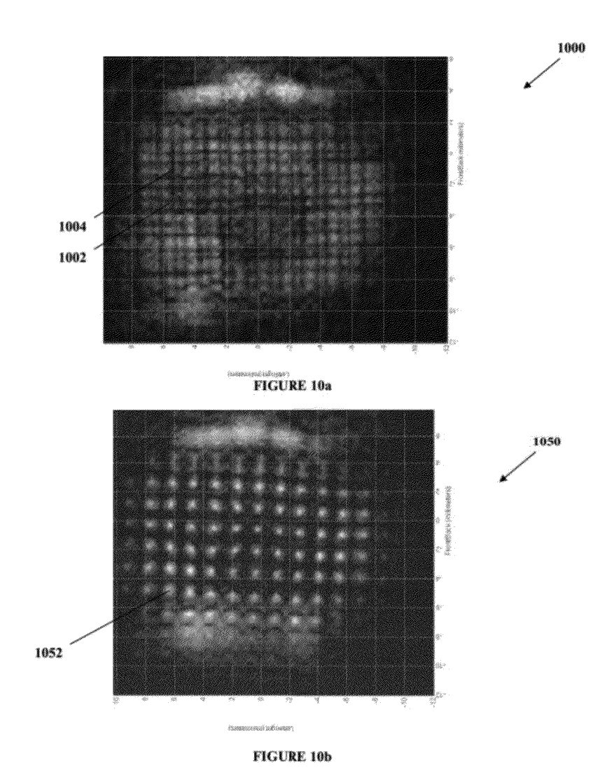

FIG. 10a illustrates a top view of the pressure field 1000 of the ultrasound transducer 910 of FIG. 9 at a distance of 9 mm from the front surface of the transducer. As with FIGS. 2a and 2b above, the shade of white represents the intensity of the pressure field. Pressure field 1000 includes regions of similar intensity 1002 separated by regions of relatively no intensity 1004. FIG. 10b illustrates a top view of the pressure field 250 of the ultrasound transducer 910 of FIG. 9 at a distance of 12 mm from the front surface of the transducer. As can be seen by a comparison of FIGS. 10a and 10b, the intensity of the field has concentrated in focal zones 1052, while the peak intensity has also increased.

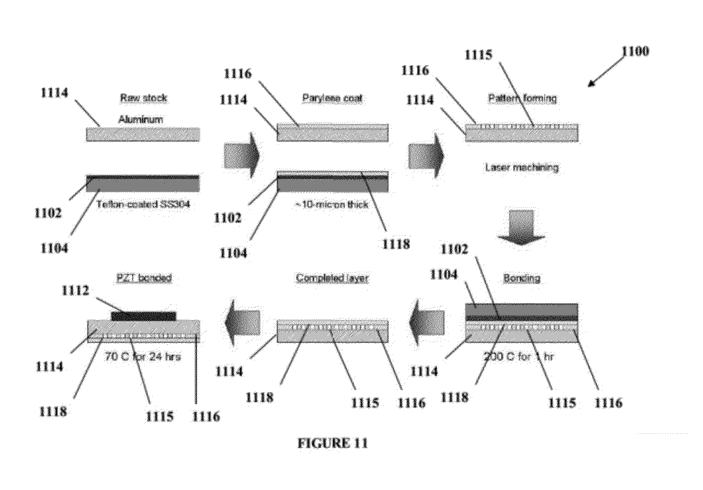

FIG. 11 illustrates another process 1100 of manufacturing an ultrasound transducer in accordance with an exemplary embodiment of the invention. It should be appreciated that process 1100 may include any number of additional or alternative tasks. The tasks shown in FIG. 11 need not be performed in the illustrated order and process 1100 may be incorporated into a more comprehensive procedure or process having additional functionality not described in detail herein.

FIG. 11 is a schematic of six steps (clockwise, starting at top-left) in the manufacturing process 1100 of an exemplary ultrasound transducer 1110. Process 1100 produces an ultrasound transducer 1110 with a PZT layer 1112, an aluminum layer 1114, a first transmissive layer 1116, and a second transmissive layer 1118. First transmissive layer 1116 includes a series of etchings 1115 configured to induce scattering of an ultrasound wave (not shown) created by the PZT. In other words, the approach here for producing an apodized beam is a coating that has a spatially varying transmission coefficient.

Although aluminum is depicted as the wearplate in FIG. 11, it will readily be understood by a person of ordinary skill in the art that any suitable metal can be used as the wearplate without deviating from the scope of the invention. Such suitable methods include, but are not limited to, biocompatible materials, such as titanium.

As shown in the first two sub-figures of FIG. 11, a first transmissive layer 1116 is added to a layer of aluminum and a second transmissive layer 1118 is added to Teflon-coated 1102 layer of stainless steel 1104. As shown in the third sub-figure, a pattern is formed on the first transmissive layer 1116 to produce a series of etchings 1115. The first and second transmissive layers 1116 and 1118 are bonded together to form a three-layer assembly: an aluminum layer 1114 bonded to a first transmissive layer 1116 with etchings 1115, which is bonded to a second transmissive layer 1118. The aluminum layer 1114 is then bonded to a PZT layer 1112. The final ultrasound transducer comprises PZT layer 1112, aluminum layer 1114, first transmissive layer 1116, and second transmissive layer 1118, wherein first transmissive layer 1116 includes a plurality of air gaps that serve to scatter an acoustic wave generated by the PZT layer 1112.

The exemplary embodiments described above allow for treatment of a broad area with a single device much faster than traditional devices and at a lower cost. It is known that lesions formed in the dermis can result in skin tightening. The devices described above can be used to treat larger areas faster than current technologies. This allows for practical treatments with significantly less pain than other technologies, while being as efficacious. Further, apodization allows for smaller focal points, so the application can stop before nerves can fire. That is, if the focal zones are small enough, they will cool down faster than the perception time for nerve cells. In addition, the fractional nature of the treatment will allow the patient to tolerate higher temperatures in the tissue since these temperatures are confined to small regions.

By operating the system in a multiple-pass mode, that is, by repeatedly translating the device across the surface of a patient's skin, a large fraction of a patient's skin can be treated with temperatures much higher than could normally be achieved in traditional tightening treatments. In addition, it can do so at a greater depth than traditional treatments. Achieving high temperature in the skin is critical to achieving a positive clinical result.

Some embodiments allow more flexibility with treatment parameters than existing technology because the device can be moved rapidly from region to region. This allows multiple treatments at lower power to build up temperature in the skin slowly, so that treatment pulses require less power and have a lower perceived pain due to a smaller temperature difference during the course of exposure.

By generating an array of focal zones, embodiments of the invention can selectively heat small volumes of tissue over a large area at a specific depth for the purpose of improving skin elasticity, wrinkle density and selectively tighten certain regions for the point of mimicking surgical skin-reduction procedures such as a facelift or eyebrow lift. The periodic array of lesions is uniform and thereby provides a low likelihood of double-treatments of an area.

In addition, it is possible that introducing more lesions with a lower power will reduce complications from procedures. The array of focal zones can also treat adipose tissue to the point of apoptosis or necrosis, or treat a larger area in less time than a single, focused device.

Embodiments described herein allow physicians to target specific layers of the patient (relative the skin treatment) with focused energy. This protects the epidermis and upper layers of the dermis, while reducing variations in efficacy due to variations in contact cooling of the skin. For example, embodiments described herein can spare the epidermis from injury during a treatment while heating the mid-to-lower dermis to over 50° C. in a single application.

The cost of manufacture is low enough that doctors could select different resonant frequencies for the patient based on the acoustic properties of their skin (attenuation) and increase the uniformity of patient-to-patient response. This is desirable for the patients to feel confident that the treatment is efficacious, desirable for a doctor to be viewed as providing a quality service, and medical suppliers as providing quality product.

Further, embodiments of the invention can be produced at a lower cost than transducers with focusing elements. In addition, a flat PZT can be used, which reduces the cost associated with shaping a PZT to produce one or more spherical waves.

Other technologies such as light, RF, and single-focused ultrasound do not offer the speed, large area of treatment, and depth selectivity of this technology. This opens the possibility for a new level of clinical efficacy relative to the risk, pain and final clinical outcome (benefit) of a treatment.

In some embodiments, endpoints for the treatment range from local gentle heating (non-apoptotic) to formation of thermal lesions. As mentioned above, embodiments of the ultrasound transducer can be tailored to generate the lesions at different depths.

In some embodiments, the ultrasound transducer is driven by an RF generator, using conventional impedance matching techniques. For treatment of patients, the output surface of the device may be held in contact with the patient either with or without the help of an intermediate coupling gel. Exposure durations may range from 1 ms to 30 seconds depending on the type of treatment. The power output of the device may be limited by the clinical endpoint of the specific treatment. The device may be used to deliver a single high-output power pulse to treat the patient, or may produce a series of smaller pulses that accumulate over time to a desirable clinical endpoint. The device may be held still (no motion relative to the patient) during the aforementioned treatments or it could be moved across the surface of the skin.

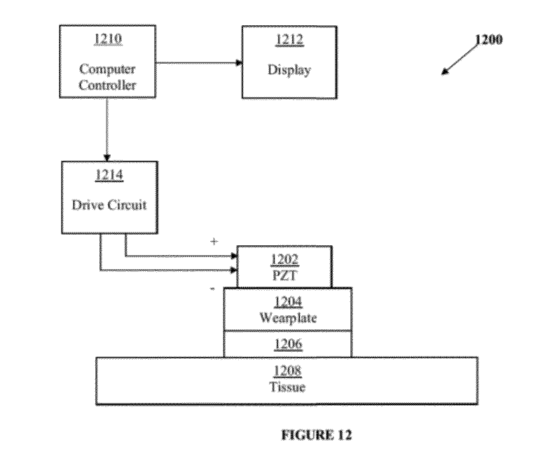

FIG. 12 illustrates an exemplary ultrasonic transducer system 1200 in accordance with an exemplary embodiment of the invention. System 1200 includes a PZT layer 1202, a wearplate 1204, a transmissive layer 1206, a computer controller 1210, a display 1212, and a drive circuit 1214. System 1200 is placed on a patient's tissue 1208. PZT layer 1202, wearplate 1204, and transmissive layer 1206 may comprise any of the embodiments described herein. Additional or less layers may be incorporated in system 1200 without deviating from the scope of the invention.

A drive circuit 1214 is used to produce the excitation voltage for the one or more PZT layer 1202. As shown in FIG. 12, the drive circuit may drive the PZT using a pair of electrodes. The drive circuit 1204 may be a waveform generation device suitable for delivering an ultrasonic frequency voltage. In some embodiments, more than one waveform-generation device is used as the drive circuit 1204. In some embodiments, the drive circuit 1204 may be controlled by a computer controller 1202. In some embodiments, the drive circuit 1204 includes an internal controller in addition to, or instead of, computer controller 1202. In another embodiment, it is possible to set the drive circuit 1204 to more than one excitation frequency and more than one treatment duration time.

The computer controller 1202 may include one or more processors for executing computer-readable instructions. The computer-readable instructions allow the computer to control the drive circuit 1204 to produce one or more drive frequencies at one or more drive voltages. The computer controller may also include computer memory, such as read-only memory (ROM), random-access memory (RAM), and one or more non-volatile storage media drives for storing computer-readable instructions or programs. The computer controller may be equipped with a computer display 1206 or other visual read-out device.

Although the invention has been described in connection with some embodiments, it is not intended to be limited to the specific form set forth herein. Rather, the scope of the invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A device for selectively treating multiple regions of tissue simultaneously, the device comprising:
an ultrasound transducer configured to produce an acoustic wave; and
a plurality of layers associated with the ultrasound transducer, wherein at least one of the plurality of layers comprises a plurality of cavities, where the plurality of cavities are configured to scatter the acoustic wave and simultaneously produce a plurality of treatment zones at a predetermined distance from the ultrasound transducer.

2. The device of claim 1, wherein the plurality of cavities are further configured to produce at least one of the plurality of treatment zones by constructive interference of at least two portions of the acoustic wave that are scattered by a respective two of the plurality of cavities.

3. The device of claim 1, wherein the one of the plurality of layers comprises one selected from the group consisting of: a piezoelectric source, a wearplate, and a transmissive layer.

4. The device of claim 3, wherein the plurality of cavities comprises a plurality of etchings within the layer.

5. The device of claim 3, wherein the one of the plurality of layers comprises the transmissive layer and the plurality of cavities comprise a material different from a material of the transmissive layer.

6. The device of claim 1, wherein the plurality of cavities are arranged to create a pattern.

7. The device of claim 6, wherein the pattern is one selected from a hexagonal symmetry and a square symmetry.

8. The device of claim 1, wherein at least one of the plurality of cavities comprises a circular shape.

9. The device of claim 1, wherein at least one of the plurality of cavities comprises a polygonal shape.

10. The device of claim 9, wherein the polygonal shape is one selected from the group consisting of a square and a hexagon.

11. The device of claim 10, wherein the plurality of cavities are arranged to create a pattern, and wherein the pattern is one selected from a hexagonal symmetry and a square symmetry.

12. The device of claim 1, wherein at least one of a size and a position of each of the plurality of cavities is optimized for a predetermined application.

13. The device of claim 12, wherein the predetermined application comprises at least one of a predetermined skin type and a predetermined treatment.

14. The device of claim 13, wherein the predetermined treatment comprises a calculation of the predetermined distance.

15. The device of claim 13, wherein the at least one of the size and the position comprises selecting a distance between cavities.

16. An ultrasound device for selectively treating multiple regions of tissue simultaneously comprising:
an ultrasound transducer assembly comprising a planar delivery surface through which ultrasound energy is transmitted, the assembly comprising a planar region located between the delivery surface and the tissue and further comprising a periodic pattern of alternating higher and lower transmission regions, the planar region functioning to cause interference in ultrasonic waves and cause the ultrasound energy to be focused at multiple regions in a plane spaced from the transducer.

17. The device of claim 16, wherein the planar region is formed integrally with the transducer.

18. The device of claim 16, wherein the planar region is formed in a separate layer bonded to the transducer.

19. The device of claim 16, wherein the periodic pattern is formed by openings in the layer.
